(19) United States
(12) Patent Application Publication
(10) Pub. No.: US 2025/0144472 A1
(43) Pub. Date: May 8, 2025

(54) Method and System for Verifying an Activity Metric

(71) Applicant: Prevayl Innovations Limited, Manchester, Greater Manchester (GB)

(72) Inventors: Martin Ashby, Manchester, Greater Manchester (GB); Abbas Syed, Manchester, Greater Manchester (GB); Michael John Lynch, Cheshire (GB)

(21) Appl. No.: 18/838,310

(22) PCT Filed: Feb. 8, 2023
(86) PCT No.: PCT/GB2023/050276

§ 371 Date: Aug. 14, 2024

(30) Foreign Application Priority Data
Feb. 21, 2022 (GB) 2202289.1

(51) Int. Cl. A63B 24/00; A61B 5/0205; A61B 5/024; A61B 5/11

(52) U.S. Cl. CPC A63B 24/0062; A61B 5/0205; A63B 24/0059; A61B 5/02405; A61B 5/1118

(57) ABSTRACT

An unverified activity metric is obtained for an activity performed by a user (602). The activity metric may be the distance travelled by the user, the number of repeated actions performed by the user, or an activity intensity classification. Physiological data is obtained for the user while performing the activity (604). The physiological data may be cardiac activity data, capacitance data, temperature data, or bioimpedance. The activity metric is verified by applying a verification function that uses the physiological data to verify that the user performed the activity (606). The verification function may verify that the physiological sensor is in proximity with the user from the physiological data.

### BACKGROUND

It is desirable to incentivise exercise to encourage more healthy lifestyles. One way to do this is to provide awards in response to user participation in activities. The awards may be in the form of digital assets such as digital tokens. Other forms of awards are possible such as reductions in insurance premiums.

Existing system generate awards based on the number of steps taken by a user. For example, STEPN by FindSatoshi Lab Ltd., is a move-to-earn running application that awards digital tokens to users based on the number of steps taken. The digital tokens may be exchanged for non-fungible tokens, NFTs, associated with a particular digital asset used within the application. The transaction is stored on a blockchain.

A problem with such systems is that they are prone to user manipulation particularly when the awards have a monetary value or are otherwise highly desirable. Step count data such as recorded by a pedometer can be easily faked by, for example, shaking the pedometer.

International Patent Application Publication No. WO 2021/074612 A1 discloses a movement verification system. The system verifies whether a user movement represented by a sequence of repeated user actions, such as steps, is genuine by using a verification function that compares the unverified set of movement data for the user against a model. Tokens are generated based on verified movement data.

The existing approaches are limited to verifying activity metrics based on motion and in particular the number of repeated user actions such as steps. Moreover, as the verification is only performed using movement data for the user, it is only able to detect unsophisticated forms of cheating/manipulation.

It is an object of the present disclosure to provide improved methods and systems for verifying an activity metric for the user.

### SUMMARY

According to the present invention, there is provided method, computer-readable medium and system as set out in the appended claims. Other features of the invention will be apparent from the dependent claims, and the description which follows.

According to a first aspect of the disclosure, there is provided a computer-implemented method of verifying an activity metric. The method comprises obtaining an unverified activity metric for an activity performed by a user. The method comprises obtaining physiological data for the user while performing the activity. The method comprises verifying the activity metric by applying a verification function that uses the physiological data to verify that the user performed the activity.

Advantageously, the method uses physiological data verify that the user performed the activity and thus verify the activity metric. In this way, the method does not rely or does not solely rely on movement data recorded for the user. Physiological data is generally harder to fake/manipulate than movement data alone and therefore the method provides for more accurate activity metric verification.

The physiological data may be indicative of whether a physiological sensor is in proximity to the user. The verification function may verify that the user performed the activity based on whether the physiological data indicates that the physiological sensor is in proximity to the user.

Advantageously, the verification function is able to verify whether the physiological sensor is in proximity to the user based on the physiological data obtained. This can be used to verify that the user is actually wearing the sensor and performing the activity rather than, for example, coupling the sensor to a signal generator.

The physiological data may comprise cardiac activity data for the user.

The cardiac activity data may comprise heartrate data for the user.

The cardiac activity data may comprise heartrate variability data for the user.

The physiological data may comprise capacitance data.

The physiological data may comprise breathing rate data for the user.

The physiological data may comprise temperature data for the user.

The physiological data may comprise bioimpedance data.

The physiological data may comprise optical data.

The physiological data may comprise a combination of any of cardiac activity data, capacitance data, breathing rate data, temperature data, bioimpedance data, and optical data.

The verification function may verify that the user performed the activity based on whether the physiological data is consistent with the activity metric.

Advantageously, the verification function is able to verify that the physiological data is consistent with the activity metric. If, for example, the activity metric indicates that the user performed vigorous exercise, but the physiological data shows that the user was likely at rest, the verification function is able to identify that the activity metric was likely faked/manipulated.

The verification function may verify that the user performed the activity based on whether the physiological data is consistent with expected physiological data for the user.

Advantageously, the verification function is able to verify that the physiological data is consistent with the expected physiological data for the user. The verification function may compare the physiological data to a history of physiological data for the user and/or for population norms. The population norms may relate to physiological data obtained from different users having similar characteristics to the user such as a similar age.

The physiological data may comprise heartrate variability data for the user and where the verification function may verify that the user performed the activity based on whether the heartrate variability data is consistent with expected heartrate variability data for the user.

The verification function may verify that the user performed the activity based on whether the physiological data identifies the user.

Advantageously, the verification function is able to verify that the physiological data identifies the user. A biometric identification procedure may therefore be used which compares a biometric identify obtained from the physiological data to one or more previously obtained biometric identities for the user. The verification function is therefore able to identify whether the user is performing the activity, or the activity is being performed by a different user.

The activity metric may comprise the distance travelled by the user during the activity.

The activity metric may comprise a number of repeated actions performed by the user during the activity.

The activity metric may comprise an activity intensity classification for the user for the activity.

The method may further comprise generating an award for the user based on the activity metric.

Generating an award may comprise awarding a digital asset to the user based on the activity metric.

The method may further comprise transferring the digital asset to a digital wallet of the user.

The method may further comprise storing a record of the transfer of the digital asset in a distributed ledger.

According to a second aspect of the disclosure, there is provided a system for verifying an activity metric, the system comprises a processor and a memory, the memory storing instructions which, when executed by the processor, cause the processor to perform operations comprising obtaining an unverified activity metric for an activity performed by a user, obtaining physiological data for a user while performing the activity, and verifying the activity metric by applying a verification function that uses the physiological data to verify that the user performed the activity.

The operations may comprise any of the operations of the first aspect of the disclosure.

According to a third aspect of the disclosure, there is provided a system for verifying an activity metric. The system comprises an electronics module positionable in proximity to a user and comprising one or more physiological sensors configured to monitor one or more parameters of the user while performing an activity. The system comprises an activity verification module configured to obtain an unverified activity metric for the activity performed by the user and the physiological data, and further configured to verify the activity metric by applying a verification function that uses the physiological data to verify that the user performed the activity.

According to a fourth aspect of the disclosure, there is provided a computer-implemented method of verifying a distance travelled by a user, the method comprises obtaining an unverified distance travelled by a user during an activity, obtaining physiological data for the user while performing the activity, and verifying the distance travelled by the user by applying a verification function that uses the physiological data to verify that the user performed the activity.

The computer-implemented method may also comprise generating an award for the user based on the distance.

The method may comprise any of the features of the first aspect of the disclosure.

According to a fifth aspect of the disclosure, there is provided a system for verifying an activity metric, the system comprises a processor and a memory, the memory storing instructions which, when executed by the processor, cause the processor to perform operations comprising obtaining an unverified distance travelled by a user during an activity, obtaining physiological data for the user while performing the activity, and verifying the distance travelled by the user by applying a verification function that uses the physiological data to verify that the user performed the activity.

The operations may comprise any of the operations of the first aspect of the disclosure.

According to a sixth aspect of the disclosure, there is provided a system for verifying an activity metric. The system comprises an electronics module positionable in proximity to a user and comprising one or more physiological sensors configured to monitor one or more parameters of the user while performing an activity. The system comprises an activity verification module configured to obtain an unverified distance travelled for the activity performed by the user and the physiological data, and further configured to verify the distance travelled by the user by applying a verification function that uses the physiological data to verify that the user performed the activity.

According to a seventh aspect of the disclosure, there is provided a computer-implemented method of verifying a number of repeated actions performed by a user, the method comprises obtaining an unverified number of repeated actions performed by a user during an activity, obtaining physiological data for the user while performing the activity, and verifying the number of repeated actions performed by the user by applying a verification function that uses the physiological data to verify that the user performed the activity.

The computer-implemented method may also comprise generating an award for the user based on the number of repeated actions.

The method may comprise any of the features of the first aspect of the disclosure.

According to a eighth aspect of the disclosure, there is provided a system for verifying a number of repeated actions performed by a user, the system comprises a processor and a memory, the memory storing instructions which, when executed by the processor, cause the processor to perform operations comprising obtaining an unverified number of repeated actions performed by a user during an activity, obtaining physiological data for the user while performing the activity, and verifying the number of repeated actions performed by the user by applying a verification function that uses the physiological data to verify that the user performed the activity.

The operations may comprise any of the operations of the first aspect of the disclosure.

According to a ninth aspect of the disclosure, there is provided a system for verifying an activity metric. The system comprises an electronics module positionable in proximity to a user and comprising one or more physiological sensors configured to monitor one or more parameters of the user while performing an activity. The system comprises an activity verification module configured to obtain an unverified number of repeated actions performed by the user during the activity and the physiological data, and further configured to verify the number of repeated actions performed by the user by applying a verification function that uses the physiological data to verify that the user performed the activity.

According to a tenth aspect of the disclosure, there is provided a computer-implemented method of verifying an activity intensity classification for a user, the method comprises obtaining an unverified activity intensity classification for an activity performed by a user, obtaining physiological data for the user while performing the activity, and verifying the activity intensity classification by applying a verification function that uses the physiological data to verify that the user performed the activity.

The computer-implemented method may also include generating an award for the user based on the number of repeated actions.

The method may comprise any of the features of the first aspect of the disclosure.

Obtaining the activity intensity classification may comprise determining a maximum heart rate for the user, obtaining a time series of heart rate data representing the heart rate for the user while performing an activity, determining one or more activity intensity levels based on the measured heart rate relative to the maximum heart rate for the user, measuring the time spent at each of the one or more training intensity levels during the activity, and generating an activity intensity classification based on the relative time spent at each of the one or more training intensity levels.

According to a eleventh aspect of the disclosure, there is provided a system for verifying a number of repeated actions performed by a user, the system comprises a processor and a memory, the memory storing instructions which, when executed by the processor, cause the processor to perform operations comprising obtaining an unverified activity intensity classification for an activity performed by a user, obtaining physiological data for the user while performing the activity, and verifying the activity intensity classification by applying a verification function that uses the physiological data to verify that the user performed the activity.

The operations may comprise any of the operations of the first aspect of the disclosure.

According to a twelfth aspect of the disclosure, there is provided a system for verifying an activity metric. The system comprises an electronics module positionable in proximity to a user and comprising one or more physiological sensors configured to monitor one or more parameters of the user while performing an activity. The system comprises an activity verification module configured to obtain an unverified activity intensity classification for the activity performed by the user and the physiological data, and further configured to verify the activity intensity classification by applying a verification function that uses the physiological data to verify that the user performed the activity.

According to a thirteenth aspect of the disclosure, there is provided a computer-implemented method of verifying an activity metric, the method comprises obtaining an unverified activity metric for an activity performed by a user, obtaining contextual information for the user while performing the activity, and verifying the activity metric by applying a verification function that uses the contextual information to verify that the user performed the activity.

Advantageously, contextual information such as location information or environmental information is sued to verify that the user performed the activity. Contextual information is hard to fake/manipulate by the user particularly if multiple sources of contextual information are used. In this way, a robust method of verifying an activity metric is provided.

The contextual information may comprise signal quality information for a physiological sensor arranged to measure physiological signals for the user.

The verification function may verify that the user performed the activity based on whether the signal quality information changes over time.

The verification function may verify that the user performed the activity based on whether the signal quality information indicates that the signal quality decreases over the duration of the activity.

The contextual information may comprise location data for the user during the activity, and where the verification function uses the location data to verify that the user performed the activity.

The verification function may determine whether the location data is consistent with the activity.

The location data may comprise first location data obtained from a first source of location information for the user and second location data obtained from a second source of location information for the user, and where the verification function determines whether the first location data is consistent with the second location data.

The first source of location information may comprise a location sensor for a first device associated with the user and the second source of location information may comprise a location sensor for a second device associated with the user.

The contextual information may comprise environmental data, and where the verification function uses the environmental data to verify that the user performed the activity. The verification function may determine whether the environmental data is consistent with the activity.

The environmental data may comprise one or more of pressure data, air quality data, ambient light data, ambient humidity data, and ambient temperature data.

The contextual information may comprise location data, and where the verification function determines whether the environmental data is consistent with the location data.

The environmental data may comprise barometric pressure data, and where the verification function determines whether the amplitude data derived from the barometric pressure data is consistent with amplitude data derived from the location data.

The method may comprise any of the features of the first aspect of the disclosure.

According to a fourteenth aspect of the disclosure, there is provided a system for verifying an activity metric, the system comprises a processor and a memory, the memory storing instructions which, when executed by the processor, cause the processor to perform operations comprising obtaining an unverified activity metric for an activity performed by a user, obtaining contextual information for the user while performing the activity, and verifying the activity metric by applying a verification function that uses the contextual information to verify that the user performed the activity.

The operations may comprise any of the operations of the fourteenth aspect of the disclosure.

According to a fifteenth aspect of the disclosure, there is provided a computer-readable medium having instructions recorded thereon which, when executed by a processor, cause the processor to perform the method of the first, fourth, seventh or tenth aspect of the disclosure.

### BRIEF DESCRIPTION OF THE DRAWINGS

To easily identify the discussion of any particular element or act, the most significant digit or digits in a reference number refer to the figure number in which that element is first introduced.

FIG. 1 illustrates an example activity verification system according to aspects of the present disclosure.

FIG. 2 illustrates an example page of an application according to aspects of the present disclosure.

FIG. 3 illustrates an example page of an application according to aspects of the present disclosure.

FIG. 4 illustrates an example page of an application according to aspects of the present disclosure.

FIG. 5 illustrates an example page of an application according to aspects of the present disclosure.

FIG. 6 illustrates a flow diagram for an example method according to aspects of the present disclosure.

FIG. 7 illustrates a flow diagram for an example method according to aspects of the present disclosure.

FIG. 8 illustrates a flow diagram for an example method according to aspects of the present disclosure.

FIG. 9 illustrates a flow diagram for an example method according to aspects of the present disclosure.

FIG. 10 illustrates a flow diagram for an example method according to aspects of the present disclosure.

FIG. 11 illustrates a flow diagram for an example method according to aspects of the present disclosure.

FIG. 12 illustrates a schematic of an example electronics arrangement in accordance with aspects of the present disclosure.

FIG. 13 illustrates a schematic of an example electronics arrangement in accordance with aspects of the present disclosure.

FIG. 14 illustrates an example analogue-to-digital frontend of an electronics module according to aspects of the present disclosure.

### DETAILED DESCRIPTION

The following description with reference to the accompanying drawings is provided to assist in a comprehensive understanding of various embodiments of the disclosure as defined by the claims and their equivalents. It includes various specific details to assist in that understanding but these are to be regarded as merely exemplary. Accordingly, those of ordinary skill in the art will recognize that various changes and modifications of the various embodiments described herein can be made without departing from the scope and spirit of the disclosure. In addition, descriptions of well-known functions and constructions may be omitted for clarity and conciseness.

The terms and words used in the following description and claims are not limited to the bibliographical meanings but are merely used by the inventor to enable a clear and consistent understanding of the disclosure. Accordingly, it should be apparent to those skilled in the art that the following description of various embodiments of the disclosure is provided for illustration purpose only and not for the purpose of limiting the disclosure as defined by the appended claims and their equivalents.

It is to be understood that the singular forms “a,” “an,” and “the” include plural referents unless the context clearly dictates otherwise.

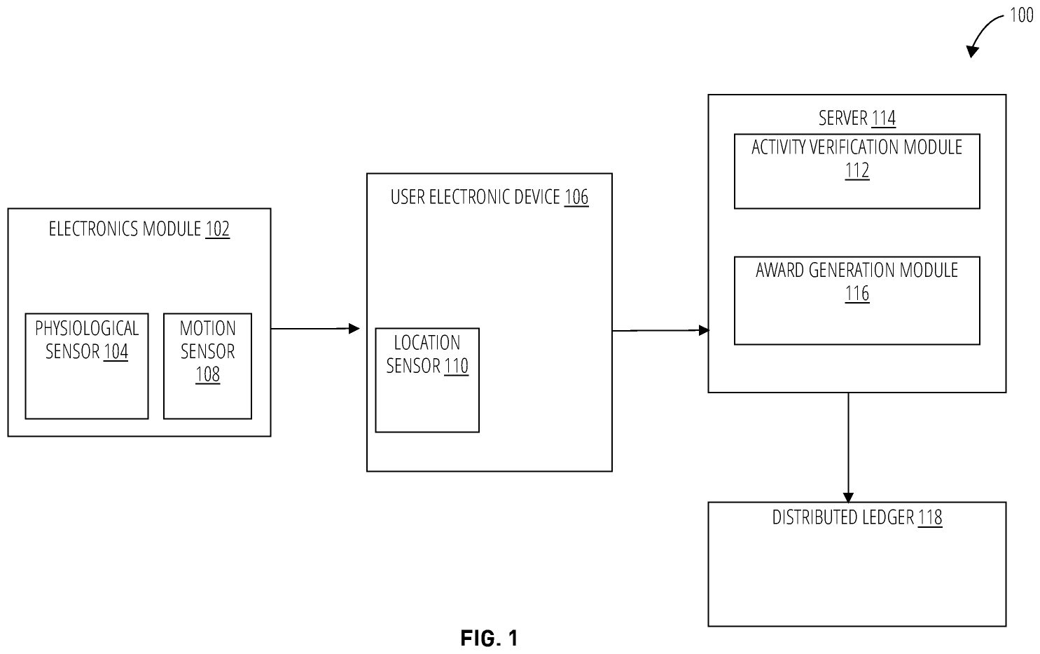

FIG. 1 shows an activity verification system 100 in accordance with aspects of the present disclosure.

The activity verification system 100 comprises an electronics module 102, user electronic device 106, and server 114.

The electronics module 102 is arranged to be worn by a user.

The electronics module 102 may form all or part of a wearable article. The wearable article may be any form of article which may be worn by a user such as a smart watch, necklace, garment, bracelet, or glasses. The wearable article may be a textile article. The wearable article may be a garment. The garment may refer to an item of clothing or apparel. The garment may be a top. The top may be a shirt, t-shirt, blouse, sweater, jacket/coat, or vest. The garment may be a dress, garment brassiere, shorts, pants, arm or leg sleeve, vest, jacket/coat, glove, armband, underwear, headband, hat/cap, collar, wristband, armband, chestband, waistband, stocking, sock, or shoe, athletic clothing, personal protective equipment, including hard hats, swimwear, wetsuit or dry suit.

The wearable article (e.g., a garment) may be constructed from a woven or a non-woven material. The wearable article may be constructed from natural fibres, synthetic fibres, or a natural fibre blended with one or more other materials which can be natural or synthetic. The yarn may be cotton. The cotton may be blended with polyester and/or viscose and/or polyamide according to the application. Silk may also be used as the natural fibre. Cellulose, wool, hemp and jute are also natural fibres that may be used in the wearable article. Polyester, polycotton, nylon and viscose are synthetic fibres that may be used in the wearable article.

The garment may be a tight-fitting garment or a loose-fitting (e.g., freeform garment). A tight-fitting garment helps ensure that the electrodes of the garment are held in contact with or in the proximity of a skin surface of the wearer. The tight-fitting garment may be a compression garment. The tight-fitting garment may be an athletic garment such as an elastomeric athletic garment. A loose-fitting garment is generally more comfortable to wear over extended time periods and during sleep.

The electronics module 102 comprises a physiological sensor 104 and, in this example, a motion sensor 108. The motion sensor 108 is not required in all examples. The motion sensor may comprise one or more of an accelerometer and a gyroscope.

The physiological sensor 104 monitors one or more physiological properties of the user. The one or more physiological properties are indicative of whether the electronics module 102 is in proximity to the user.

The physiological sensor 104 may comprise a biopotential sensor such as an ECG sensor. Biopotential sensors measures the potential across the skin surface.

The physiological sensor 104 may comprise a bioimpedance sensor. Bioimpedance sensors typically comprises a source of current and a receiver. The bioimpedance sensor can measure the opposition to electric current through a part of the body of the user. The bioimpedance sensor typically measures electrical resistance and/or reactance. Example bioimpedance sensors include impedance plethysmography sensors which can be used to measure the breathing activity of the user.

The physiological sensor 104 may comprise a cardiac activity sensor such as an ECG sensor, a photoplethysmography (PPG) sensor, a ballistocardiogram (BCG) sensor or an electromagnetic cardiogram sensor. The physiological data may be in the form of heartbeat data samples for the user representative of the heartbeat activity of the user.

The physiological sensor may be an optical sensor. An optical sensor may measure the amount of ultraviolet, visible, and/or infrared light in the environment. The optical sensor may comprise a photoplethysmographic (PPG) sensor. PPG sensors measure blood volume changes within the microvascular bed of the wearer's tissue. PPG sensors use a light source to illuminate the tissue. Photodetectors within the PPG sensor measure the variations in the intensity of absorbed or reflected light when blood perfusion varies.

The physiological sensor 104 may comprise a capacitive sensor. The capacitive sensor is arranged to detect a change in capacitance value based on the proximity of the user's skin to the capacitive sensor. This can be used to determine whether the electronics module 102 is being worn by the user. The physiological data may be in the form of capacitance data.

The physiological sensor 104 may comprise a temperature sensor. The temperature sensor may be arranged to measure a temperature of the user. The temperature sensor may be a contact temperature sensor or a non-contact temperature sensor such as an infrared temperature sensor. The physiological data may be in the form of temperature data.

The physiological sensor 104 may comprise an audio sensor such as a microphone. The audio sensor may be arranged to measure sound properties of the user such as cardiac sounds.

The physiological sensor 104 may couple to electrodes provided as part of the wearable article. This may be used for biopotential sensors or bioimpedance sensors for example. The electrodes are typically provided on an inside surface of the wearable article and are held in close proximity to a skin surface of the user.

The electrodes may be made of a (electrically) conductive material such as a conductive yarn, conductive ink, conductive transfer, or conductive paste. When formed from conductive yarn, the electrodes may be knitted, woven, embroidered, stitched or otherwise incorporated into the wearable article. The electrodes may be integrally formed with the wearable article such as by being integrally knitted with the wearable article. A signal set of electrodes may be shared by multiple sensors such as an ECG sensor and a bioimpedance sensor.

The electronics module 102 is not required to couple to electrodes in a wearable article and may also be a stand-alone component without communicatively coupling to a wearable article.

The electronics module 102 is typically removably coupled to the wearable article such that it is retained by the wearable article when worn. The electronics module 102 can be removed from the wearable article so that the wearable article can be washed without damaging the internal electronics of the electronics module 102. The electronics module 102 can also be removed from the wearable article for charging. In other examples, the electronics module 102 is integrally formed with the wearable article such as when the wearable article/electronics module form a smartwatch.

The electronics module 102 communicates physiological data and motion data to the user electronic device 106. The physiological data communicated with depend on the type of physiological sensor incorporated into the electronics module 102.

The user electronic device 106 is communicatively coupled to the electronics module 102 such that it can send data to and receive data from the electronics module 102.

The user electronic device 106 may be in in the form of a mobile phone or tablet and may comprise components such as a processor, a memory, a wireless communicator, a display, a user input unit, a capturing device in the form of a camera, and a motion sensor. The user electronic device 106 in this example also comprises a location sensor to monitor the location of the user.

The user electronic device 106 is configured to execute an application. The application is configured to establish a communication session with the electronics module 102. This may involve the user electronic device 106 prompting the user to tap their user electronic device 106 against the electronics module 102 to trigger a pairing process between the user electronic device 106 and the electronics module 102.

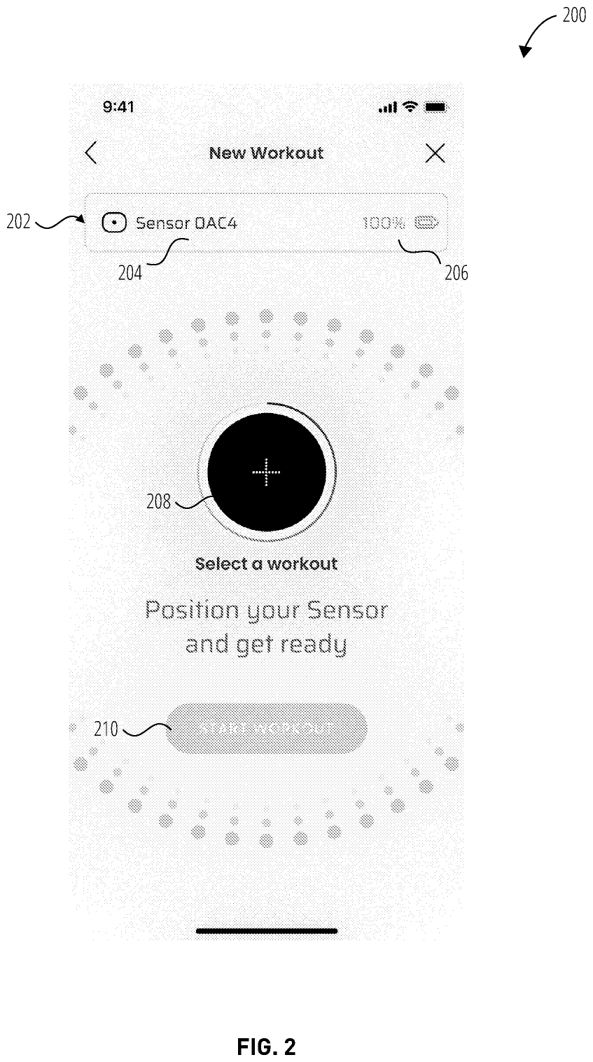

Once a communication session has been established with the electronics module 102, the user is taken to page 200 as shown in FIG. 2.

The page 200 displays connection information 202 for the electronics module 102 connected to the user electronic device 106. The connection information 202 includes identity information 204 for the electronics module 102 and a battery status 206 of the electronics module 102.

The page 200 additionally displays a button 208 for allowing a user to select a type of workout being performed. Example workouts that may be selected include: outdoor cycling, indoor cycling, mountain biking, outdoor running, exercise on a treadmill, interval training, weight training, high intensity interval training, walking, hiking, yoga/pilates, and rowing.

The page 200 additionally displays a button 210 to start the workout. The button 210 is disabled as the workout has not yet been selected by the user.

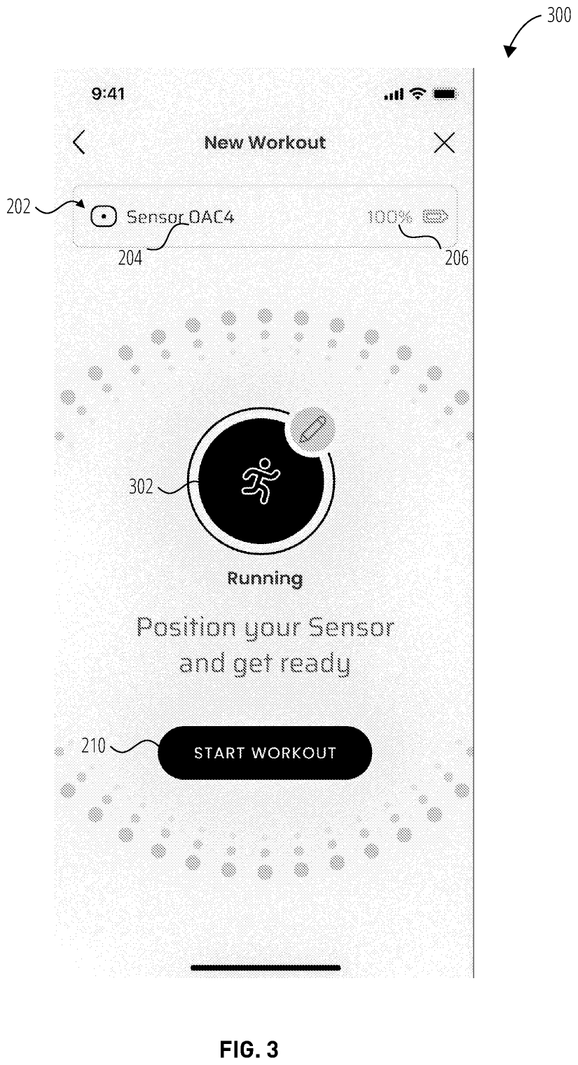

In response to the user selecting a workout via button 208, the application transitions to page 300 as shown in FIG. 3. The page 300 displays an information element 302 indicating the workout that the user has selected. In this example, the user has selected running.

The page 300 additionally displays the connection information 202 and button 210 to start the workout. The button 210 is now enabled. Once the button 210 is selected, the workout starts. It will be appreciated that the user does not have to manually select to being an activity/stop an activity in all examples. The application may automatically detect the onset of an activity based on data received from the electronics module 102. This could be based on an activity classification received from the electronics module 102 which indicates that the user has transitioned from a resting state to an active state. Likewise, an end of an activity could be detected based on data received from the electronics module 102. This could be based on an activity classification received from the electronics module 102 which indicates that the user has transitioned from an active state to a resting state.

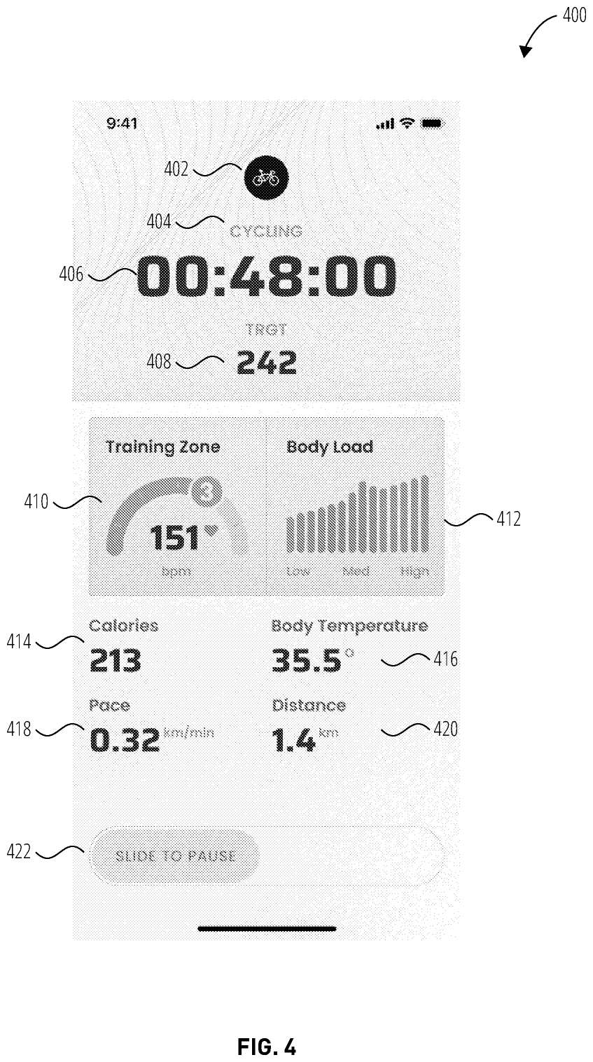

FIG. 4 shows an example page 400 displayed on the user electronic device 106 during the activity (a workout in this example). In this example, the user is performing an outdoor cycling activity as indicated by information element 402 and text element 404.

The page 400 displays a timer 406 showing the amount of time that has elapsed during the workout.

During the workout, the user electronic device 106 receives physiological data from the electronics module 102. In this example, the physiological data includes heartrate data and, in particular, a sequence of heartbeat data samples for the user representative of the heartbeat activity of the user over the time span. The heartbeat data samples comprising inter-beat interval, IBI, values representing the time between successive heartbeats. In this example, the IBI values are calculated by the controller 1210 of the electronics module 102 from digital signal values received from the analogue-to-digital front end 1416.

The page 400 displays an activity intensity classification 408 (TRGT score) that is determined based on the time that the user has spent in different activity intensity levels. The activity intensity levels are determined from the heartrate of the wearer which is derived from the IBI values.

The page 400 also displays a visual indicator 410 of the activity intensity level that the user is currently in.

The page 400 also displays a visual indicator 412 of the current body load of the user. The body load is derived from the determined body temperature of the user. The body temperature may be determined from temperature measurements performed by a temperature sensor of the electronics module 102 and/or may be derived from the heartbeat data samples.

The page 400 also displays other indicators including a calorie consumption indicator 414, a body temperature indicator 416, a pace indicator 418, and a distance travelled indicator 420.

The page 400 also includes a pause toggle 422 to allow the user to pause the workout.

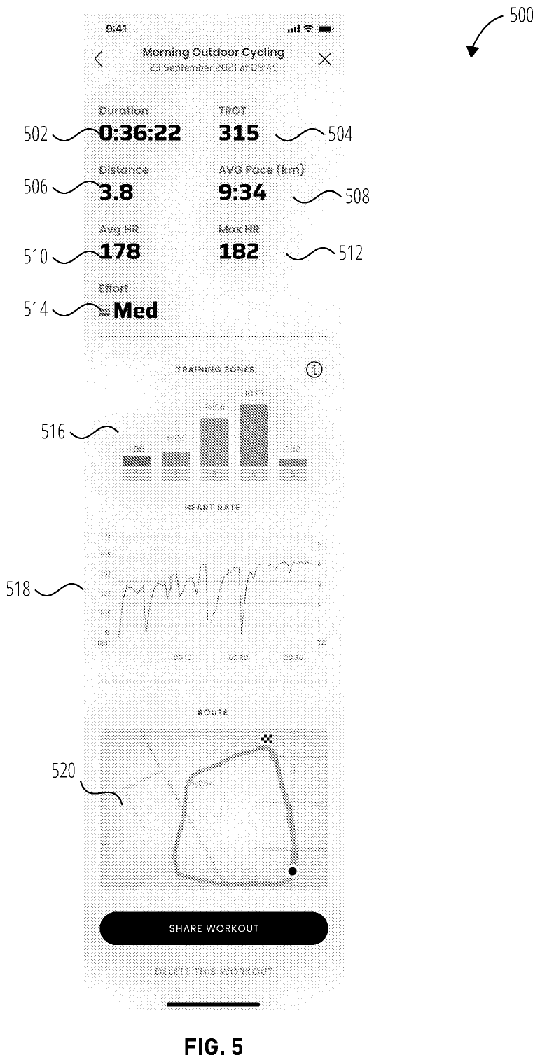

FIG. 5 shows an example workout summary page 500 that is displayed after the user completes their workout. The workout summary page 500 displays a number of activity metrics that summarise the performance of the user during the workout.

The activity metrics include a duration 502 of the workout, an activity intensity classification 504 of the workout, a distance travelled 506 for the workout, an average pace 508 for the workout, an average heartrate 510 for the workout, a maximum heartrate 512 for the workout, and an effort level 514 for the workout.

The page 500 additionally displays a visual indicator of the time in different training zones 516, a visual indicator of the heartrate over time 518, and a visual indicator of the route travelled 520.

To increase engagement and incentivise exercise, it is desirable to generate awards for the user based on their activity metrics. The award may be generated based on one or more of the duration 502, activity intensity classification 504, distance travelled 506, average pace 508, average heartrate 510, maximum heartrate 512, or effort level 514. The award may be generated based on other activity metrics not shown in FIG. 5 such as the number of repeated actions performed by the user. The repeated actions may be, for example, the number of steps or bicycle revolutions. The award may be generated to incentivise certain behaviours. For example, awards may be generated based on the user regularly performing on-demand testing using the application. This may be a form of repeated action performed once or several times each day. For example, the user may receive an award based on performing an activity on each of a predetermined number of consecutive days. The award may be generated based on other activity metrics such as the breathing rate of the user and ventilatory thresholds of the user.

The awards may be in the form of digital assets such as digital coins or digital tokens. The digital token may be a non-fungible token, NFT, which may be associated with a specific digital media item.

The digital asset may be a cryptographically secured digital asset such as a computer-generated digital/virtual collectable. The digital asset may be secured and/or uniquely identified by a digital, cryptographic, token and may be linked and/or distributed with a digital model of the asset.

The digital asset may be any form of computer-generated virtual object including digital footwear, apparel, headgear, avatars, pets etc., that may have a unique non-fungible token (NFT) registered on a blockchain or otherwise registered in an immutable database. The user may be able to sell, trade, or exchange the digital asset for physical, fiat and/or digital currency. A digital online marketplace may be maintained that includes an inventory of digital assets for sale.

The digital asset may be capable of being imported into one or more other digital platforms to serve, for example, as a skin on a video game or virtual world character that may be developed and/or controlled by the user. In some examples, different attributes of the digital asset may impart changes in the ability level of the user's character.

The awards may take other forms such as a reduction in an insurance premium or other forms of benefits. Other forms of award are within the scope of the present disclosure.

The awards may have a monetary value or may otherwise be highly desirable. Users may attempt to cheat or exploit the system to acquire the awards. Cheating may be attempted by trying to manipulate the electronics module. Cheating may involve artificially moving the electronics module 102 to mimic repeated user actions without having the user actually perform the user actions. Cheating may involve providing artificial location data to the user electronic devices 106 through techniques such as GPS spoofing to give the appearance that the user has travelled a certain distance. Cheating may involve feeding artificially generated signals (e.g., computer generated or pre-recorded) ECG signals to the electronics module 102 to mimic high intensity cardiac activity. Multiple users may also use the same electronics module 102 to give the appearance that a single user is performing activities.

The present disclosure uses the physiological data to verify that the user actually performed the activity to therefore verify that the activity metric is genuine. The verification generally involves verifying that the electronics module is in proximity to the user and not coupled to a signal generator, for example.

The user electronic device 106 provides the unverified activity metric and the physiological data to the server 114. The physiological data provided to the server 114 may be a processed version of the data received from the electronics module 102. The user electronic device 106 may for example determine one or more features from the physiological data provide these metrics to the server 114.

The server 114 is in communication with the user electronic device 106 such that it can send data to and receive data from the user electronic device 106. The server 114 may not necessarily be in the form of a single physical machine. The term “server” may encompass, for example, a distributed computing arrangement.

The server 114 comprises an activity verification module 112. The activity verification module 112 is arranged to verify the activity metric determined by the user electronic device 106. The activity verification module 112 deploys a verification function that uses the physiological data to verify if the activity was performed by the user. This enables the activity verification system 100 to determine whether the activity metric was generated based on real or manipulated/fake data.

The physiological data may comprise capacitance data indicative of whether a capacitive sensor of the electronics module 102 is in proximity to the user. The verification function may verify that the activity metric genuine if the capacitance data indicates that the electronics module 102 is in proximity to the user. The capacitance sensor will record different capacitance values based on whether it is in proximity to a skin surface of a living being as compared to an inanimate object. In this way, the capacitance data can be used to verify whether the electronics module 102 is coupled to a signal generator instead of an actual user.

The physiological data may comprise temperature data. The verification function may verify that the activity metric is genuine if the temperature data indicates that the electronics module 102 is in proximity to the user. The temperature sensor will record different temperatures based on whether it is in proximity to a skin surface of a living being as compared to an inanimate object. In this way, the temperature data can be used to verify whether the electronics module 102 is coupled to a signal generator instead of an actual user.

The verification function may also look at the variation in temperature over time. It would generally be expected that the temperature of the user would increase when performing high intensity activities and decrease when at rest. If the activity metric indicates that the user performed strenuous exercise, but the temperature did not increase accordingly, then the verification function may verify that the activity is not genuine. Similarly, if the temperature increases over time, the verification function may verify that the activity is genuine.

The physiological data may comprise bioimpedance data. The verification function may verify that the activity metric is genuine if the bioimpedance data indicates that the electronics module is in proximity to the user. The bioimpedance sensor will record different bioimpedance values based on whether it is in proximity to a skin surface of a living being as compared to an inanimate object. In this way, the bioimpedance data can be used to verify whether the electronics module 102 is coupled to a signal generator instead of an actual user.

The bioimpedance data may be indicative of the breathing rate of the user over time. It would be expected that the breathing rate of the user is not constant and would increase when performing high intensity activities and decrease when at rest. If the activity metric indicates that the user performed strenuous exercise, but the breathing rate data did not increase accordingly, then the verification function may verify that the activity is not genuine. Similarly, if the breathing rate increases over time, the verification function may verify that the activity is genuine.

The physiological data may comprise cardiac activity data such as user heart rate data and/or heartrate variability data. The verification function may verify that the activity metric is genuine if the cardiac activity data indicates that the cardiac activity has not been artificially generated and/or is consistent with the activity metric.

For example, if the activity metric indicates that the user ran for a certain distance, but the cardiac activity indicates that the user was likely at rest (e.g, a low average heartrate) then the verification function may verify that the activity metric is not genuine. Meanwhile, if the cardiac activity indicates that the user was likely exercising (e.g., a higher average heartrate) then the verification function may verify that the activity metric is genuine. The verification function may use one or more average heartrate thresholds for different user activities. The average heartrate thresholds may be user specific and may be generated based on parameters such as the age, gender, weight, and exercise history of the user. The average heartrate thresholds may additionally or alternatively be based on a history of heartrate data for the user and/or population norms.

This example can capture some examples of faked cardiac activity data but may not capture all examples such as when artificially generated or pre-recorded cardiac signals commensurate with the activity are fed to the electronics module 102. However, these types of faked cardiac activity data can also be detected by using additional checks such as via capacitance data or temperature data.

In another example, the verification function may use the heartrate variability to determine whether the activity metric is genuine. The heartrate variability (HRV) represents the variation in time intervals between consecutive heartbeats of the user. Genuine heartbeats are not normally evenly spaced in time, but instead vary. An artificially generated cardiac signal may typically be expected to have no time variation between heartbeats. The verification function may therefore identify such artificially generated cardiac signals by comparing the measured HRV to a threshold level. A number of different threshold levels may be used for different user activities. The threshold levels may be user specific and may be generated based on parameters such as the age, gender, weight, and exercise history of the user. The threshold levels may additionally or alternatively be based on a history of HRV data for the user and/or population norms.

The verification function may use a combination of the different types of physiological data as described above. For example, a combination of one or more of capacitance data, temperature data, bioimpedance data, and cardiac activity data may be used to verify that the user performed the activity.

In some examples, the verification function may use the physiological data to verify the identity of the user. The physiological data in this example is data that uniquely identifies the user. This may be, for example, physiological data that cardiac activity data.

The physiological sensor used for user identification may be an optical sensor such as a PPG sensor. PPG signals measured by a PPG sensor can be used to uniquely identify a wearer because unique characteristics of the wearer's vascular system lead to unique features being present in the PPG signal. The second derivative of PPG signals (SDPPG) may also be used to uniquely identify a person as SDPPG signals vary from person to person.

The physiological sensor used for user identification may be an electrical sensor such as an ECG sensor or bioimpedance sensor. An electrical sensor may measure the electrical activity of a part of the body or how a current changes which it is applied to the body. An electrical sensor may perform biopotential measurements. An example biopotential sensor is an electrocardiaogram, ECG, sensor that measures the electrical activity of the heart. A user's heartbeat may be analysed using patterns gathered by the ECG sensor, which records a heart's electric potential changes in time. A longer recording of heartbeat activity is called an electrocardiogram (ECG) and is recorded using one or more pairs of electrodes. The change of electrical potential is measured between the points of contact of the electrodes. This change is strongly correlated with heart and muscle activity of the subject as the heartbeat activity of the human body is stimulated through electrical impulses. An electrical sensor may perform bioimpedance measurements. That is, the electrical sensor may comprise a bioimpedance sensor. Bioimpedance measurements may be obtained by performing different impedance measurements between different points on user's body at different frequencies. An example bioimpedance sensor is a galvanic skin response sensor that measures the skin conductance. The skin conductance varies depending on the amount of moisture (induced by sweat) in the skin. Sweating is controlled by the sympathetic part of the nervous system, so it cannot be directly controlled by the subject. The skin conductance can be used to determine body response against physical activity, stress, or pain. The body response against these stimuli differ from person to person and so can be used to uniquely identify the wearer of the electronics module.

The physiological sensor used for user identification may comprise a temperature sensor such as a skin temperature sensor. A skin temperature sensor may comprise a thermopile arranged to capture infrared energy and transform it into an electrical signal that represents the temperature. The skin temperature may be unique to the user, and in particular may vary in a unique or predictable way in response to physical activity, stress, or pain.

The physiological sensor used for user identification may comprise an acoustic sensor. The acoustic sensor may comprise a microphone. The acoustic sensor may be arranged to measure the user's voice. The user's voice is defined by the physiological characteristics of their respiratory system and can be used to uniquely identify the user. In addition, other properties such as the vocabulary, style, syntax, and other features of speech also identify the user and can be determined from the captured audio signal. The acoustic sensor may be arranged to measure other (typically low power) sounds emitted from the user, such as the user's heart. Therefore, the acoustic sensor can measure heartbeat sounds which can be used to define the heart rate variability or other uniquely identifying property of the user wearing the electronics module.

A combination of different types of sensors may be used to uniquely identify the user.

In some examples, the verification function may additionally use the motion data to verify if the activity is genuine. The motion data may be used to verify if the motion of the user is genuine rather than artificially generated such as by the user shaking the electronics module 102.

The motion data may be used to obtain an activity classification for the user. The activity classification may identify the type of activity performed by the user according to the motion sensor (e.g., standing, walking, running, or cycling). The activity classification can be compared to the activity metric as part of the process of verifying that the activity is genuine. For example, if the activity metric indicates that the user performed a certain number of repeated actions (e.g., steps) but the activity classification indicates that the user was standing, then the verification function can identify that the activity metric is not genuine.

In some examples, the verification function comprises a machine-learned model trained based on verified data for a user or group of users. The verified data may be obtained in a controlled environment. The physiological data or features extracted from the physiological data may be input to the machine-learned model to verify if the activity metric is genuine. Other data such as motion data for the user may be input to the machine-learned model.

The server 114 further comprises an award generation module 116. If the activity verification module 112 determines that the activity is genuine, the award generation module 116 generates an award for the user based on the activity metric.

In some examples, the award may be in the form of a digital asset. The digital asset may be transferred to a digital wallet of the user and the transaction may be recorded in distributed ledger 118.

In some examples, the verification algorithm will consider multiple factors to verify that the user performed the activity. These may include a combination of: verifying that the electronics module is in proximity with the user; verifying that the physiological data identifies the user; and verifying that the physiological data is consistent with the activity metric.

FIG. 1 just shows one preferred arrangement of the activity verification system 100. It will be appreciated that the user electronic device 106 is not required in all examples as the electronics module 102 may communicate directly with the server 114. It will also be appreciated that the activity verification module 112 is not required to be located on the server 114 in all examples and may be located on the user electronic device 106 or the electronics module 102. The user electronic device 106 and the electronics module 102 may also be embodied as a single device.

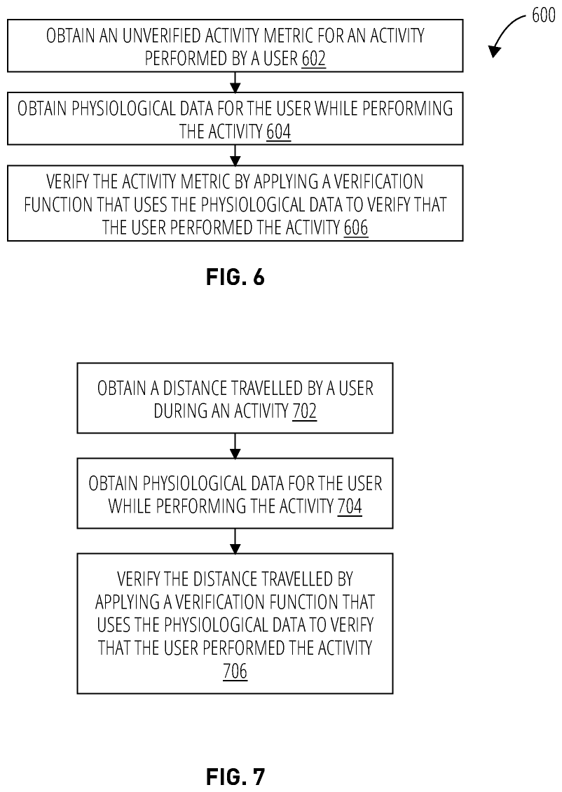

FIG. 6 shows an example flow diagram of a method for verifying an activity metric. The method may be performed by the activity verification system 100 described above.

Step 602 comprises obtaining an unverified activity metric for an activity performed by a user. Step 604 comprises obtaining physiological data for the user while performing the activity. Step 606 comprises verifying the activity metric by applying a verification function that uses the physiological data to verify that the user performed the activity.

FIG. 7 shows an example flow diagram of a method for verifying an activity metric. The method may be performed by the activity verification system 100 described above.

In this example, the activity metric is the distance travelled by the user during an activity. The distance travelled may be obtained from location data recorded by the location sensor 110 of the user electronic device 106. The location data is susceptible to spoofing.

Step 702 comprises obtaining the distance travelled by the user during the activity. Step 704 comprises obtaining physiological data for the user while performing the activity. Step 706 comprises verifying the distance travelled by the user by applying a verification function that uses the physiological data to verify that the user performed the activity.

The verification function may verify that the user performed the activity if the physiological data indicates that the electronics module was in proximity to the user, for example. The verification function may additionally use motion data, or an activity classification derived from the motion data to verify that the user was performing the activity. The verification function may additionally or separately use the physiological data to verify that physiological data is consistent with the activity metric and/or that the physiological data identifies the user.

The method may further comprise generating an award for the user based on the distance travelled by the user. This could simply involve awarding one or more digital tokens based on the total distance travelled. Alternatively or additionally a digital asset could be awarded if the user travels a certain distance each day or cumulatively over a number of days.

In some examples, the award is generated based on an estimated emissions saving for the user as compared to travelling a corresponding distance using a vehicle. Example emissions include carbon dioxide. This can act to reward users for walking, running or cycling to work as compared to travelling by a vehicle such as a car, bus or train. This helps to incentivise environmentally friendly practices.

The award generation module 116 may calculate an estimate of the emissions saved from the distance travelled by the user and a metric that represents the amount of emissions (e.g., carbon dioxide) if the distance were travelled by a vehicle. For example, a car may be expected to emit between 170 and 260 grams of carbon dioxide per kilometre. The award generation module 116 may select use a value within this range and multiple the value by the distance travelled to determine the emissions saving. A digital asset such as a token may be award based on the emissions saved.

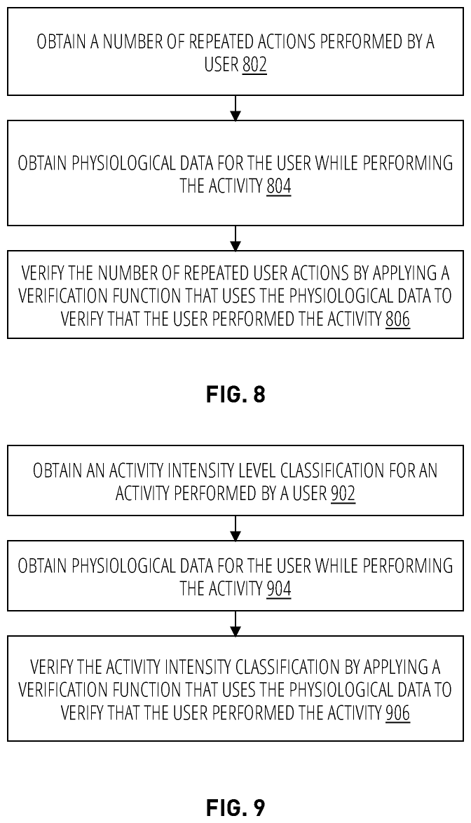

FIG. 8 shows an example flow diagram of a method for verifying an activity metric. The method may be performed by the activity verification system 100 described above.

In this example, the activity metric is the number of repeated actions performed by a user such as the number of steps taken by the user. The number of steps may be determined by a motion sensor of the electronics module 102. The motion sensor may comprise a pedometer for calculating the number of steps from accelerometer and/or gyroscope data.

Step 802 comprises obtaining a number of repeated actions performed by a user during an activity. Step 804 comprises obtaining physiological data for the user while performing the activity. Step 806 comprises verifying the number of repeated actions performed by the user by applying a verification function that uses the physiological data to verify that the user performed the activity.

The verification function may verify that the user performed the activity if the physiological data indicates that the electronics module was in proximity to the user, for example. The verification function may additionally use motion data or an activity classification derived from the motion data to verify that the user was performing the activity. The verification function may additionally or separately use the physiological data to verify that physiological data is consistent with the activity metric and/or that the physiological data identifies the user.

The method may further comprise generating an award for the user based on the number of repeated actions performed by the user. This could simply involve awarding one or more digital tokens based on the total number of repeated actions. Alternatively or additionally a digital asset could be awarded if the user performs a certain number of repeated actions each day or cumulatively over a number of days.

FIG. 9 shows an example flow diagram of a method for verifying an activity metric. The method may be performed by the activity verification system 100 described above.

In this example, the activity metric is an activity intensity activity intensity classification for an activity performed by a user. Generally, an activity intensity classification is determined from the heart rate of the user. Typically, the higher the heart rate, the more intense the workout. As such a measure of a user's heart rate, whilst working out, provides an indication of the intensity of the workout.

People have a resting heart rate, which is the heart rate when a person is at rest. There is also a maximum heart rate (MHR) which is the highest heart rate that the cardiovascular system can handle during physical activity. Between these two values are different zones or ranges, measured as a percentage of the maximum heart rate. These zones or ranges or often referred to as training zones and can be used as a measure of training intensity.

There are a number of different formats of training zones used during exercise to guide training levels and workouts. One that is often used divides exercise intensity into five training zones:
Zone 1: Very light, 50 percent to 60 percent of MHR
Zone 2: Light, 60 percent to 70 percent of MHR
Zone 3: Moderate, 70 percent to 80 percent of MHR
Zone 4: Hard, 80 percent to 90 percent of MHR
Zone 5: Very hard, 90 percent to 100 percent of MHR

Advice by health bodies in the US, UK and Australia is that people should aim to spend at least 150 minutes a week doing a moderate intensity workout.

Step 902 comprises obtaining the activity intensity classification for the activity performed by a user.

Determining the activity intensity classification may comprise determining a maximum heartrate (MHR) for the user. A person's MHR can be determined using known principles. The most common way to determine a person's maximum heart rate is by using one of the many age-based equations. The most well-known of these is the Fox formula: 220−age=Maximum Heart Rate (MHR)

Other age-based formulae include the Gelish equation of 207−(0.7×age) and the Tanaka equation of 208−(0.7×age). The Gelish and Tanaka equations are sometimes preferred as they have a lower standard deviation.

The MHR may be calculated using an age-based formula such as the following equation:

For females:

$$MHR = \frac{190.2}{1 + {e}^{0.0453 \times ( age - 107.5 )}}$$

For males:

$$MHR = \frac{203.7}{1 + {e}^{0.033 \times ( age - 104.3 )}}$$

where e=Euler's number=2.718282

In some examples, the MHR is determined by having the user perform exercise and measure their highest heart rate. The exercise may be maximal effort exercise which is typically performed in a controlled setting although it is also possible to determine MHR during freely performed exercise. Example methods of determining MHR during freely performed exercise are disclosed in European Patent Publication No. EP3656304.

Determining the activity intensity classification may comprise obtaining a time series of heartrate data representing the heart rate for the user while performing an activity. These may be obtained from the physiological data sensed by the electronics module as described above. The time series of heartrate data may be in the form of inter-beat interval (IBI) values.

Determining the activity intensity classification may further comprise determining one or more activity intensity levels based on the measured heart rate relative to the maximum heart rate for the user. The activity intensity levels may be defined based on the MHR of the user as described above in relation to the training zones.

Determining the activity intensity classification may further comprise measuring the time spent at each of the one or more activity intensity levels during the activity. The heartrate data is used to determine the heartrate of the user at different times during the activity and thus the activity intensity level that the user was in at this time. The total amount of time in each activity intensity level is determined for the activity. A visual representation of this can time in different training zones 516 indicator in FIG. 5.

Determining the activity intensity classification may further comprise generating an activity intensity level classification based on the relative time spent at each of the one or more activity intensity levels. The different intensity levels may have different weightings associated with them. In a simple example, the time in training zone 1 may be multiplied by a factor of 1, the time in training zone 2 may be multiplied by a factor of 2, the time in training zone 3 may be multiplied by a factor of 3, the time in training zone 4 may be multiplied by a factor of 4, and the time in training zone 5 may be multiplied by a factor of 5.

The time may be in minutes and seconds for example. Other scaling factors may be used. The scaled time in the different training zones are summed together to generate the activity intensity classification.

Step 904 comprises obtaining physiological data for the user while performing the activity. Step 906 comprises verifying the activity intensity classification by applying a verification function that uses the physiological data to verify that the user performed the activity.

The verification function may verify that the user performed the activity if the physiological data indicates that the electronics module was in proximity to the user, for example. The verification function may additionally use motion data, or an activity classification derived from the motion data to verify that the user was performing the activity. The verification function may additionally or separately use the physiological data to verify that physiological data is consistent with the activity metric and/or that the physiological data identifies the user.

The method may further comprise generating an award for the user based on the activity intensity classification. This could simply involve awarding one or more digital tokens based on the activity intensity classification. Alternatively or additionally a digital asset could be awarded if the user achieves a threshold activity classification each day or cumulatively over a number of days.

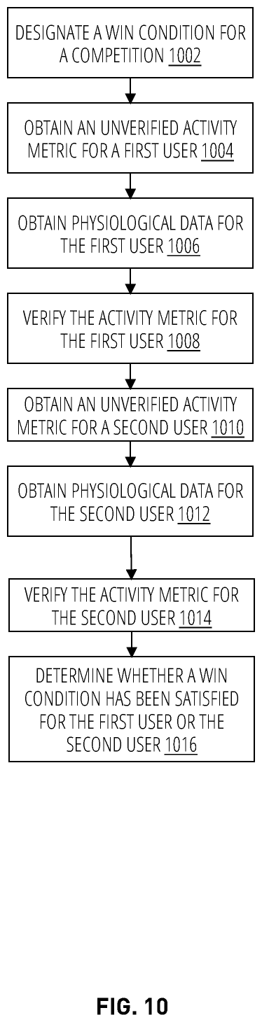

FIG. 10 shows an example flow diagram of a method for verifying an activity metric. The method may be performed by the activity verification system 100 described above.

In this example, the activity metric verification is performed in a competition environment where a plurality of users are competing to satisfy a win condition. The win condition is determined from the activity metrics.

The competition may be a race. The race may be a virtual race between multiple users in different real-world locations. The users may compete virtually through a digital environment. The users may race together at different times or at the same time. The race may be a foot race or a cycling race for example. The race may make use of exercise equipment such as treadmills, rowing machines, or cycling machines. The race may be a race from a starting line to a finishing line.

The win condition may be satisfied by the user that is the first to travel a predetermined distance. The win condition may be satisfied by the user that travels a predetermined distance in the fastest time. This could include travelling from a starting line to a finishing line. The starting line and finishing lines may be virtual.

The win condition may be satisfied by the user that travels the greatest distance during a predetermined time period. For example, the win condition may be the user that travels the fastest distance during a 10 minute condition.

The win condition may be satisfied by the user with the highest activity intensity classification during a predetermined time period. For example, the win condition may be the user with the highest activity intensity classification during a 10 minute competition.

The present disclosure is not limited to these examples and other forms of physical and virtual competitions are possible.

Step 1002 comprises designating a win condition for a competition involving at least a first user and a second user engaged in an activity. Step 1004 comprises obtaining an unverified activity metric for the activity performed by the first user. Step 1006 comprises obtaining physiological data for the first user while performing the activity. Step 1008 comprises verifying the activity metric for the first user by applying a verification function that uses the physiological data to verify that the first user performed the activity. Step 1010 comprises obtaining an unverified activity metric for the activity performed by the second user. Step 1012 comprises obtaining physiological data for the second user while performing the activity. Step 1014 comprises verifying the activity metric for the second user by applying a verification function that uses the physiological data to verify that the second user performed the activity. Step 1016 comprises determine whether the win condition has been satisfied by the first user or the second user based on the verified activity metrics.

The verification function may verify that the first or second user performed the activity if the physiological data indicates that the electronics module was in proximity to the first or second user, for example. The verification function may additionally use motion data or an activity classification derived from the motion data to verify that the first or second user was performing the activity. The verification function may additionally or separately use the physiological data to verify that physiological data is consistent with the activity metric and/or that the physiological data identifies the first or second user.

The method may further comprise generating an award for the user device that satisfies the win condition.

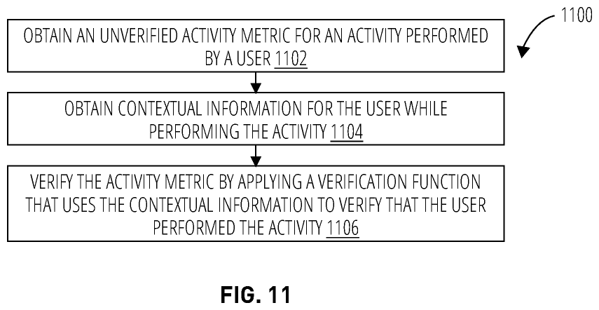

FIG. 11 shows a flow diagram for an example method according to aspects of the present disclosure. The method is performed to verify an activity metric for a user. The method may be performed by the activity verification system 100 described above.

Step 1102 comprises obtaining an unverified activity metric for an activity performed by a user.

Step 1104 comprises obtaining contextual information for the user while performing the activity.

Step 1106 comprises verifying the activity metric by applying a verification function that uses the contextual information to verify that the user performed the activity.

In this example, contextual information is used to verify the activity metric. The contextual information may be used separately or in addition to the physiological data and/or motion data as described above.

The contextual information may provide information about the environment of the user or operation of the physiological sensor when performing the activity. This information can be used to verify that the user is actually performing the activity. This information is challenging to spoof/manipulate.

In some examples, the contextual information comprises signal quality information for a physiological sensor arranged to measure physiological signals for the user. The verification function may verify that the user performed the activity based on whether the signal quality information changes over time. It would typically be expected that the signal quality will decrease over time during an activity as the user sweats. The added moisture caused by the user sweating can degrade the contact between the physiological sensor and the skin surface of the wearer and cause the wearable article to move more readily relative to the skin surface.

The verification function can consider the change in signal quality over time during the activity. If the signal quality is varying and, in particular, decreases over the duration of the activity, then the verification function can verify that the user performed the activity. If the user attempted to manipulate the activity metric by using a signal generator the change in signal quality would typically not be present or would be identifiable as artificial. The activity verification system may have a model of how the signal quality would be expected to vary for a user and/or for a type of activity and the signal quality information obtained for the activity may be compared to the model to determine if the user performed the activity.

In some examples, the contextual information comprises location data for the user during the activity. The location data may be obtained from a location sensor of the user electronic device and or the electronics module. The location sensor may be a Global Navigation Satellite System (GNSS) receiver. In some examples, the location data may be obtained from identifying the network that the electronics module and/or user electronic device is connected to. This may be obtained from WIFI SSID scans or form cellular network data.

The verification function may identify whether the location data is consistent with the activity. If the location data indicates that the user remained in the same location during the activity then the verification function may identify that the user did not perform the activity. For example, the activity metric may identify that the user performed an outdoor activity, but the location data may indicate that the user remained indoors or remained connected to one WIFI network or base station.

In some examples, the location data comprises first location data obtained from a first source of location information for the user and second location data obtained from a second source of location information for the user. The verification function may determine whether the first location data is consistent with the second location data.

For example, the electronics module and the user electronic device may both have a GNSS receiver. The location data recorded by the electronics module may be the first location data and the location data recorded by the user electronic device may be the second location data. The user may attempt to manipulate the activity metric by introducing faked location data to the user electronic device. This faked location data may indicate that the user went on a 5 km run. However, the first location data recorded by the electronics module may indicate that the user remained at home. The verification function compares the first location data and the second location data and is able to identify that they are not consistent and thus that the user did not perform the activity.

For example, the electronics module and the user electronic device may both have wireless communicators that performed look up scans to identify connection information. This could be in the form of WIFI SSID scans or base station scans in a cellular network. The verification function may compare the network identifiers recorded by the electronics module and the user electronic device and identify whether they are consistent. If the network identifier recorded by the electronics module indicates that the user remained in one location but the network identifier recorded by the user electronic device indicates that the user moved through different locations then the verification function is able to identify that the user did not perform the activity.

For example, the electronics module may have a wireless communicator and the user electronic device may have a GNSS receiver. The verification function may compare the network identifier(s) recorded by the electronics module and the GNSS data recorded by the user electronic device and identify whether they are consistent. If the network identifier recorded by the electronics module indicates that the user remained in one location but the GNSS data recorded by the user electronic device indicates that the user moved through different locations then the verification function is able to identify that the user did not perform the activity. In other examples, the user electronic device may record network identifiers and the electronics module may comprise the GNSS receiver.

In some examples, the contextual information comprises environmental data. The verification function uses the environmental data to verify that the user performed the activity. The verification function may determine whether the environmental data is consistent with the activity metric.

The environmental data relates to the environment that the user is in. The environmental data may be recorded by one or more environmental sensors of the electronics module and/or user electronic device.

The environmental sensor may comprise a light sensor arranged to detect the ambient light level. The verification function may compare the ambient light level to the activity metric to identify whether the user performed the activity. For example, if the activity metric indicates that the user went on an outdoor run, then verification function may compare the recorded ambient light level to expected ambient light levels for the time of day and weather conditions in the location of the user. The verification function may additionally or separately consider whether the ambient light level changes over time as would be expected when the user is outdoors as compared to an indoor environment where the ambient light level would be expected to be constant.

The environmental sensor may comprise a pressure sensor arranged to detect changes in ambient pressure. The pressure sensor may be a barometric pressure sensor. The verification function may compare the pressure data to the activity metric to identify whether the user performed the activity. For example, if the activity metric indicates that the user went on a hike, then the verification function may compare the barometric pressure data to expected barometric pressure data for the location where the user performed the activity. The verification function may look for pressure changes associated with the user increasing or decreasing in altitude.

The verification function may use location data along with the barometric pressure data to identify that the user performed the activity. The verification function may determine whether the barometric pressure data is consistent with the location data. If the location data indicates that the user was at a certain altitude then this may be compared to the barometric pressure data to confirm whether the user was at the altitude.

The environmental sensor may comprise an air quality sensor. The air quality sensor may comprise a carbon dioxide sensor. The air quality sensor may comprise a volatile organic compound sensor. The air quality sensor may comprise a pollen sensor.

The verification function may compare the air quality data to the activity metric to identify whether the user performed the activity. For example, if the activity metric indicates that the user went on a run in a city, then the verification function may compare the air quality data to expected air quality data for the location where the user performed the activity. The verification function may look for air quality data changes associated with the user moving through different environments such as between city parks and busy roads. The verification function may obtain the expected air quality data from a meteorology service.

The environmental sensor may comprise an ambient humidity sensor. The verification function may compare the humidity data to the activity metric to identify whether the user performed the activity. For example, if the activity metric indicates that the user went on a hike, then the verification function may compare the humidity data to expected humidity data for the location where the user performed the activity. The verification function may look for humidity changes associated with the user moving through different environments. The verification function may obtain the expected humidity data from a meteorology service.

The environmental sensor may be an ambient temperature sensor. The verification function may compare the temperature data to the activity metric to identify whether the user performed the activity. For example, if the activity metric indicates that the user went on a hike, then the verification function may compare the temperature data to expected temperature data for the location where the user performed the activity. The verification function may look for temperature changes associated with the user moving through different environments. The verification function may obtain the expected temperature data from a meteorology service.

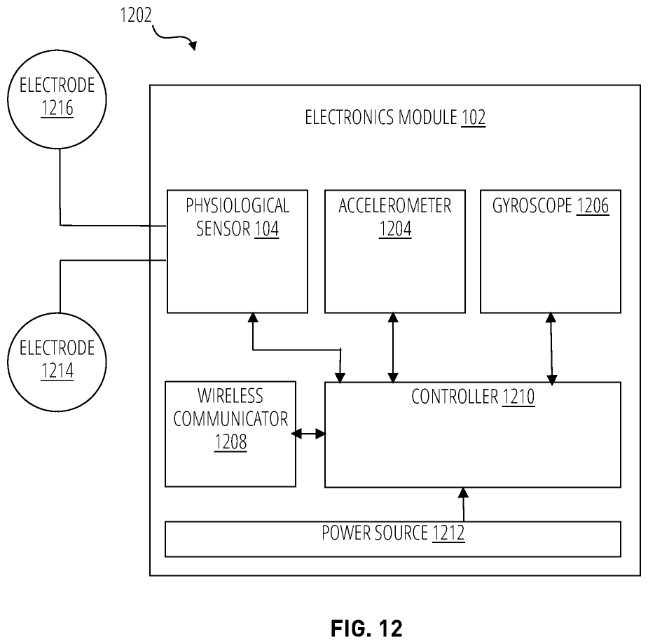

FIG. 12 shows an example electronics arrangement 1202 for a wearable article in accordance with aspects of the present disclosure. The electronics arrangement 1202 comprises an electronics module 102. The electronics module 102 comprises a controller 1210, a physiological sensor 104, an accelerometer 1204, a gyroscope 1206, a wireless communicator 1208, and a power source 1212 for supplying power to the components of the electronics module 102.

The controller 1210 comprises a processor and a memory. The controller 1210 controls the operation of the electronics module 102. The controller 1210 may comprises a plurality of processors. The controller 1210 may comprise an application processor and a machine learning processor (e.g., a machine learning core). The components of the controller 1210 may be distributed in the electronics module 102 and are not required to be provided in a single integrated circuit package.

The physiological sensor 104 communicatively couples to electrodes 1214, 1216 incorporated into the wearable article. The electrodes 1214, 1216 are placed in contact with a skin surface of a user. The physiological sensor 104 receives analogue signals from the electrodes 1214, 1216 and converts the analogue signals into digital signal values. The physiological sensor 104 may also perform additional processing on the signals such as for noise reduction.

The accelerometer 1204 and gyroscope 1206 may be provided together in an inertial measurement unit although this is not required in all examples. The gyroscope 1206 and optionally the accelerometer 1204 are controllable to transition between different power states.

The wireless communicator 1208 is arranged to communicatively couple with an external device over one or more wireless communication protocols. The wireless communication protocol may be a Bluetooth® protocol, Bluetooth® 5 or a Bluetooth® Low Energy protocol but is not limited to any particular communication protocol. The wireless communicator 1208 enables communication between the external device and the controller 1210 for configuration and set up of the controller 1210 and the peripheral devices as may be required. Configuration of the controller 1210 and peripheral devices utilises the Bluetooth® protocol in this example.

Other wireless communication protocols can also be used, such as used for communication over: a wireless wide area network (WWAN), a wireless metro area network (WMAN), a wireless local area network (WLAN), a wireless personal area network (WPAN), Bluetooth® Low Energy, Bluetooth® Mesh, Thread, Zigbee, IEEE 1402.15.4, Ant, a Global Navigation Satellite System (GNSS), a cellular communication network, or any other electromagnetic RF communication protocol. The cellular communication network may be a fourth generation (4G) LTE, LTE Advanced (LTE-A), LTE Cat-M1, LTE Cat-M2, NB-IoT, fifth generation (5G), sixth generation (6G), and/or any other present or future developed cellular wireless network.

The power source 1212 may comprise one or a plurality of power sources. The power source 1212 may be a battery. The battery may be a rechargeable battery. The battery may be a rechargeable battery adapted to be charged wirelessly such as by inductive charging. The power source 1212 may comprise an energy harvesting device. The energy harvesting device may be configured to generate electric power signals in response to kinetic events such as kinetic events performed by the wearer of the wearable article. The kinetic event could include walking, running, exercising or respiration of the wearer. The energy harvesting material may comprise a piezoelectric material which generates electricity in response to mechanical deformation of the converter. The energy harvesting device may harvest energy from body heat of the wearer. The energy harvesting device may be a thermoelectric energy harvesting device. The power source 1212 may be a super capacitor, or an energy cell.

The electronics module 102 may additionally comprise a power receiving interface operable to receive power from an external power store for charging the power source 1212. The power receiving interface may be a wired or wireless interface. A wireless interface may comprise one or more wireless power receiving coils for receiving power from the external power store.

The power receiving interface may also be coupled to the controller 1210 to enable direct communication between the controller 1210 and an external device if required.

The accelerometer 1204 monitors the acceleration of the wearer (user) of the electronics module 102. The gyroscope 1206 monitors the orientation of the wearer.

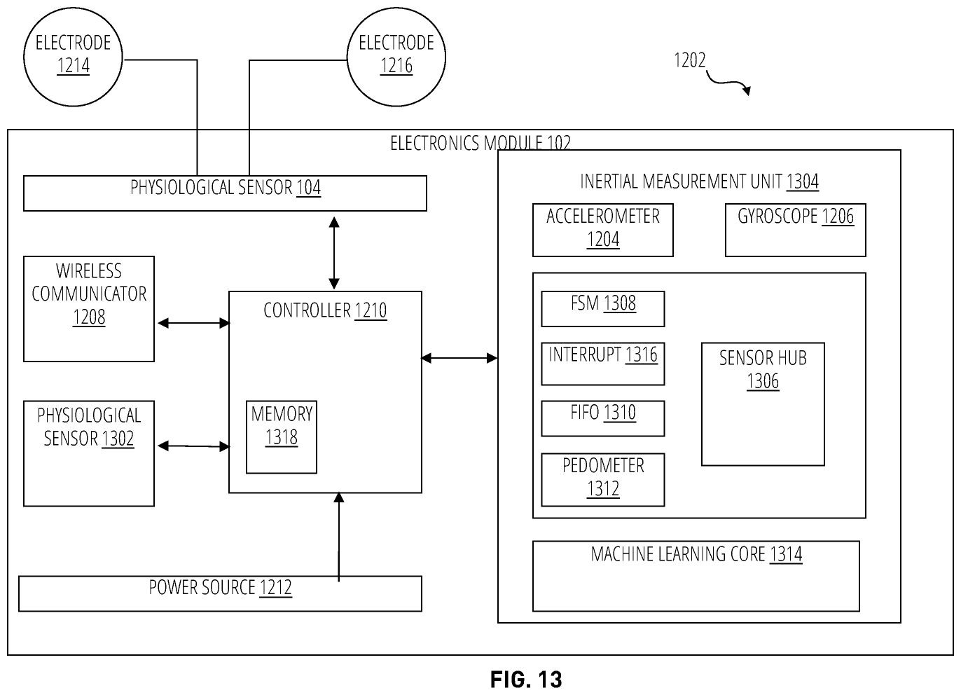

FIG. 13 shows another example electronics arrangement 1202 for a wearable article. The electronics arrangement 1202 is similar to the electronics arrangement 1202 of FIG. 13 and like reference numerals are used to indicate like components.

In this example, the accelerometer 1204 and gyroscope 1206 are provided as part of an inertial measurement unit 1304. Known examples of inertial measurement units 1304 that can be used for this application include the ST LSM6DSOX manufactured by STMicroelectronics. This inertial measurement unit 1304 a system-in-package IMU featuring a 3D digital accelerometer and a 3D digital gyroscope. Another example of a known inertial measurement unit 1304 suitable for this application is the LSM6DSO also be STMicroelectronics.

The inertial measurement unit 1304 can include machine learning functionality, for example as provided in the ST LSM6DSOX. The machine learning functionality is implemented in a machine learning processor referred to as the machine learning core 1314. The machine earning processing capability may use decision-tree logic.

The machine learning core 1314 is an embedded feature of the inertial measurement unit 1304 and comprises a set of configurable parameters and decision trees. As is understood in the art, a decision tree is a mathematical tool composed of a series of configurable nodes. Each node is characterized by an “if-then-else” condition, where an input signal (represented by statistical parameters calculated from the data sensed by the accelerometer 1204 and/or gyroscope 1206) is evaluated against a threshold.

Decision trees are stored and generate results in the dedicated output registers. The results of the decision tree can be read from the application processor at any time. Furthermore, there is the possibility to generate an interrupt for every change in the result in the decision tree, which is beneficial in maintaining low-power consumption.

Decision trees can be generated using a known machine learning tool such as Waikato Environment for Knowledge Analysis software (Weka) developed by the University of Waikato or using MATLAB® or Python™.

The decision-trees may be stored in a memory of the electronics module 102 such as an internal memory 1318 of the controller 1210 or a separate memory of the electronics module 102. The inertial measurement unit 1304 may load relevant decision-trees for activity classification based from a memory of the electronics module 102.

The machine learning core 1314 may receive accelerometer data from the accelerometer 1204 and may classify the activity of the user using the decision-trees. The classification may be output to the controller 1210. For example, the machine learning core 1314 may output the following classifications: 0=lying down, 1=sitting, 2=standing, 3=walking, 4=running, 5=jumping, 6=cycling). Other activity classifications are of course possible.

The inertial measurement unit 1304 further comprises a finite state machine (FSM) 1308, an interrupt 1316, a First-In First-Out (FIFO) buffer 1310, a pedometer 1312 (for step counting), and an application processor referred to as the sensor hub 1306. The sensor hub 1306 controls the operation of the inertial measurement unit 1304 in response to signals received from the controller 1210.

The electronics modules 102 in this example further comprises a physiological sensor 1302 in addition to the physiological sensor 104. The physiological sensor 1302 may also be for monitoring physiological properties of the user and may be referred to as a physiological sensor 1302. The physiological sensor 1302 does not couples to electrodes of the wearable article. Instead, the physiological sensor 1302 may be an optical sensor, environmental sensor, or temperature sensor for example.

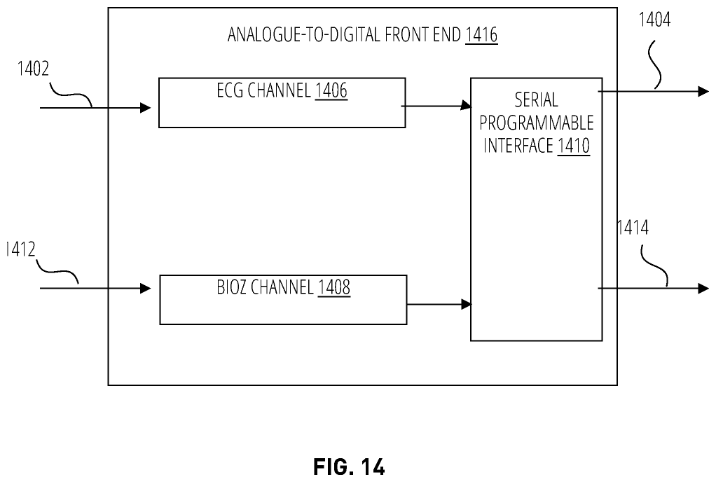

In the example of FIG. 9, the physiological sensor 104 couples to electrodes 1214, 1216 of the wearable article. The physiological sensor 104 may be in the form of an analogue-to-digital front end 1416 that couples signals received from the electrode 1214, electrode 1216 to the controller 1210. An example analogue-to-digital frontend is shown in detail in FIG. 14.

In the example described herein, the analogue-to-digital front end 1416 is an integrated circuit (IC) chip which converts the raw analogue biosignal into a digital signal for further processing by the controller 1210. ADC IC chips are known, and any suitable one can be utilised to provide this functionality. ADC IC chips for ECG and bioimpedance applications include, for example, the MAX30001 chip produced by Maxim Integrated Products Inc.

The analogue-to-digital front end 1416 includes an input 1402 and an output 1404.

Raw biosignals are input to the analogue-to-digital front end 1416, where received signals are processed in an ECG channel 1406 and a bioimpedance (BIOZ) channel 1408 and subject to appropriate filtering through high pass and low pass filters for static discharge and interference reduction as well as for reducing bandwidth prior to conversion to digital signals. The reduction in bandwidth is important to remove or reduce motion artefacts that give rise to noise in the signal due to movement of the electrodes 1214, 1216.

The output digital signals may be decimated to reduce the sampling rate prior to being passed to a serial programmable interface 1410 of the analogue-to-digital front end 1416. Signals are output to the controller via the serial programmable interface 1410.

The digital signal values output to the controller 1210 are stored in a FIFO data buffer. The controller 1210 performs operations to generate biological metrics from the digital signal values. The operations are performed in real-time while the ADC front end 139 are outputting new digital signals to the controller 1210.

ADC front end IC chips suitable for ECG applications may be configured to determine information from the input biosignals such as heart rate and the QRS complex and including the R-R interval. Alternatively, the determination of such inter-beat interval (IBI) values can be determined by the controller 1210.

The determining of the QRS complex can be implemented for example using the known Pan Tomkins algorithm as described in Pan, Jiapu; Tompkins, Willis J. (March 1985). “A Real-Time QRS Detection Algorithm”. IEEE Transactions on Biomedical Engineering. BME-32 (3): 230-236.

The controller 1210 can also be configured to apply digital signal processing (DSP) to the digital signal from the analogue-to-digital front end 1416.

The DSP may include noise filtering additional to that carried out in the analogue-to-digital front end 1416 and may also include additional processing to determine further information about the signal from the analogue-to-digital front end 1416.

At least some of the example embodiments described herein may be constructed, partially or wholly, using dedicated special-purpose hardware. Terms such as ‘component’, ‘module’ or ‘unit’ used herein may include, but are not limited to, a hardware device, such as circuitry in the form of discrete or integrated components, a Field Programmable Gate Array (FPGA) or Application Specific Integrated Circuit (ASIC), which performs certain tasks or provides the associated functionality. In some embodiments, the described elements may be configured to reside on a tangible, persistent, addressable storage medium and may be configured to execute on one or more processors. These functional elements may in some embodiments include, by way of example, components, such as software components, object-oriented software components, class components and task components, processes, functions, attributes, procedures, subroutines, segments of program code, drivers, firmware, microcode, circuitry, data, databases, data structures, tables, arrays, and variables.

Although the example embodiments have been described with reference to the components, modules and units discussed herein, such functional elements may be combined into fewer elements or separated into additional elements. Various combinations of optional features have been described herein, and it will be appreciated that described features may be combined in any suitable combination. In particular, the features of any one example embodiment may be combined with features of any other embodiment, as appropriate, except where such combinations are mutually exclusive. Throughout this specification, the term “comprising” or “comprises” means including the component(s) specified but not to the exclusion of the presence of others.

All of the features disclosed in this specification (including any accompanying claims, abstract and drawings), and/or all of the steps of any method or process so disclosed, may be combined in any combination, except combinations where at least some of such features and/or steps are mutually exclusive.

Each feature disclosed in this specification (including any accompanying claims, abstract and drawings) may be replaced by alternative features serving the same, equivalent or similar purpose, unless expressly stated otherwise. Thus, unless expressly stated otherwise, each feature disclosed is one example only of a generic series of equivalent or similar features.

The invention is not restricted to the details of the foregoing embodiment(s). The invention extends to any novel one, or any novel combination, of the features disclosed in this specification (including any accompanying claims, abstract and drawings), or to any novel one, or any novel combination, of the steps of any method or process so disclosed.

1-23. (canceled)

24. A computer-implemented method of verifying an activity metric, the method comprising:
obtaining an unverified activity metric for an activity performed by a user;
obtaining physiological data for the user while performing the activity; and
verifying the activity metric by applying a verification function that uses the physiological data to verify that the user performed the activity.

25. The method of claim 24, wherein the physiological data is indicative of whether a physiological sensor is in proximity to the user, and wherein the verification function verifies that the user performed the activity based on whether the physiological data indicates that the physiological sensor is in proximity to the user.

26. The method of claim 24, wherein the physiological data comprises cardiac activity data for the user.

27. The method of claim 24, wherein the physiological data comprises heart rate data for the user.

28. The method of claim 24, wherein the physiological data comprises heart rate variability data for the user.

29. The method of claim 24, wherein the physiological data comprises capacitance data for the user.

30. The method of claim 24, wherein the physiological data comprises breathing rate data for the user.

31. The method of claim 24, wherein the physiological data comprises temperature data for the user.

32. The method of claim 24, wherein the physiological data comprises bioimpedance data for the user.

33. The method of claim 24, wherein the verification function verifies that the user performed the activity based on whether the physiological data is consistent with the activity metric.

34. The method of claim 24, wherein the verification function verifies that the user performed the activity based on whether the physiological data is consistent with expected physiological data for the user.

35. The method of claim 24, wherein the physiological data comprises heartrate variability data for the user and wherein the verification function verifies that the user performed the activity based on whether the heartrate variability data is consistent with expected heartrate variability data for the user.

36. The method of claim 24, wherein the verification function verifies that the user performed the activity based on whether the physiological data identifies the user.

37. The method of claim 24, wherein the activity metric comprises the distance travelled by the user during the activity.

38. The method of claim 24, wherein the activity metric comprises a number of repeated actions performed by the user during the activity.

39. The method of claim 24, further comprising
generating an award for the user based on the activity metric;
wherein generating an award comprises awarding a digital asset to the user based on the activity metric.

40. The method of claim 39, further comprising
transferring the digital asset to a digital wallet of the user; and
storing a record of the transfer of the digital asset in a distributed ledger.

41. A computer-readable medium having instructions recorded thereon which, when executed by a processor, cause the processor to perform the method of claim 24.

42. A system for verifying an activity metric, the system comprising a processor and a memory, the memory storing instructions which, when executed by the processor, cause the processor to perform operations comprising:
obtaining an unverified activity metric for an activity performed by a user;
obtaining physiological data for a user while performing the activity; and
verifying the activity metric by applying a verification function that uses the physiological data to verify that the user performed the activity.

43. A system for verifying an activity metric, the system comprising:
an electronics module positionable in proximity to a user and comprising one or more physiological sensors configured to monitor one or more parameters of the user while performing an activity;
an activity verifier configured to obtain an unverified activity metric for the activity performed by the user and the physiological data, and further configured to verify the activity metric by applying a verification function that uses the physiological data to verify that the user performed the activity.
